(19) United States
(12) Patent Application Publication
(10) Pub. No.: US 2024/0131129 A1
(43) Pub. Date: Apr. 25, 2024

(54) VACCINE FOR THERAPEUTIC OR PROPHYLACTIC TREATMENT OF MYASTHENIA GRAVIS

(71) Applicants: Nicolas HAVELANGE, Bruxelles (BE); Stéphane HUBERTY, Rixensart (BE); CURAVAC EUROPE S.A., Rixensart (BE)

(72) Inventors: Nicolas HAVELANGE, Bruxelles (BE); Stéphane HUBERTY, Rixensart (BE)

(73) Assignee: CURAVAC EUROPE S.A., Rixensart (BE)

(21) Appl. No.: 18/278,985

(22) PCT Filed: Feb. 28, 2022
(86) PCT No.: PCT/EP2022/054909

§ 371 Date: Aug. 24, 2023

(30) Foreign Application Priority Data
Feb. 26, 2021 (BE) 2021/5140

(51) Int. Cl. A61K 39/00; A61P 25/28; A61P 37/06

(52) U.S. Cl. CPC A61K 39/0008; A61P 25/28; A61P 37/06; A61K 2039/6037

(57) ABSTRACT

Pharmaceutical composition for treating myasthenia gravis, comprising a carrier protein being SEQ ID NO:1 coupled to a plurality of a peptide epitope, the corresponding peptide epitopes and the method of synthesis of the conjugate.

### TECHNICAL FIELD

The present invention relates to the treatment of myasthenia gravis by immunization (vaccination) by means of an antigen receptor mimetic.

### PRIOR ART

Myasthenia gravis (MG) is a neuromuscular disorder that is characterized by a state of weakness and exhaustion of the skeletal muscles.

The underlying cause of this disease is a decrease in the number of acetylcholine receptors at the level of the (postsynaptic) neuromuscular junctions. This is caused by attack of the immune system by specific antibodies of this receptor. In fact, these antibodies, on binding to the receptor, will reduce the number of the latter at the cell surface. These antibodies will also reduce the capacity of the receptor for binding acetylcholine, and will even cause damage to the postsynaptic muscle membrane. These antibodies are in the IgG class and are dependent on the T lymphocytes.

The manifestations of the clinical symptoms of MG are correlated with the presence of these pathogenic antibodies at the neuromuscular junction.

To date, unfortunately, there are few therapies. These aim firstly to relieve the symptoms. However, such therapies are onerous, and require a strict protocol to be followed for long periods. Their efficacy is often only partial, which means that the patients' lifestyle is still affected.

The ideal treatment of MG consists of selective destruction of the pathogenic components of the immune response, i.e. the antibodies against the acetylcholine receptor.

In this context, therapeutic vaccination (immunization) by means of an antigen receptor mimetic is promising.

Patent EP2004217 describes such an approach: two complementary peptides of the acetylcholine receptor are coupled by glutaraldehyde via their —NH2 terminal end to the carrier protein keyhole limpet haemocyanin (KLH). One of the peptides aims at the production of anti-idiotypic antibodies by the patient, so as to neutralize the pathogenic antibodies and the immune response based on the B lymphocytes, and the other aims to neutralize the response based on the T lymphocytes; these two peptides therefore produce a synergistic response.

However, the clinical response, although encouraging, would benefit from being further improved.

Patent application WO 2016/184963 describes grafting of short hydrophilic hexapeptides derived from glycoprotein gp41 of the AIDS virus on protein CRM-197 or on KLH, in the hope of generating an effective vaccine against this disease. Crosslinking is carried out directly or by means of a heterobifunctional agent. Although several theoretical peptide/CRM-197 ratios are mentioned, no complete example describes said implementation, especially for peptides that are more complex or less hydrophilic.

Patent application EP2659906 describes an epitope of alpha-synuclein and also suggests CRM-197 carrier protein or KLH, but does not describe coupling of peptide epitopes on CRM-197.

The approach based on immunization (vaccination) by means of complementary peptides is further complicated by the harmful effects that may be caused by the carrier proteins and the adjuvants, since high antibody titres are required.

Each potential composition must in addition be tested in a suitable animal model, which is complicated and expensive and moreover raises ethical questions, so that this type of study is not possible if its sole purpose is the screening of new compositions. Studies conducted on rodents have shown the efficacy of one or other complementary peptide coupled to a carrier protein without the need to administer both peptides to obtain a therapeutic effect on induced myasthenia, whereas in dogs, with natural myasthenia, a synergistic effect of the two aforementioned peptides was identified and judged indispensable for obtaining a rapid and lasting therapeutic effect.

### BRIEF SUMMARY OF THE INVENTION

A first aspect of the present invention is a pharmaceutical composition comprising a peptide epitope selected from SEQ ID NO:3, SEQ ID NO:5 and/or a (synergistic) mixture of the two.

Alternatively, according to a variant, one aspect of the present invention is a pharmaceutical composition comprising one or more peptide epitopes selected from the group consisting of SEQ ID NO:2, SEQ ID NO:3, SEQ ID NO:4 and SEQ ID NO:5, this (these) peptide epitope(s) being coupled covalently to one or more free —NH2 residues of SEQ ID NO:1.

Advantageously, a plurality of one of the peptides comprising, on the one hand, SEQ ID NO:2 or SEQ ID NO:3, and/or, on the other hand, SEQ ID NO:4 or SEQ ID NO:5 is coupled to several free —NH2 residues of SEQ ID NO:1, preferably to at least 4, 5, 6, 7 or 8 free —NH2 residues of SEQ ID NO:1 and/or to fewer than 20, 19, 18, 17, 16 or 15 free —NH2 residues of SEQ ID NO:1.

Preferably, coupling was carried out by means of a heterobifunctional crosslinking agent, advantageously a crosslinking agent that is reactive for an amine group and a sulphydryl group, preferably sulpho-GMBS.

Preferably, this pharmaceutical composition is for immunization (vaccination) of a patient, preferably selected from the group consisting of human (Homo sapiens), dog (Canis vulgaris), horse (Equus caballus) and a member of the camel family (Camelus sp.), advantageously for use in the treatment of myasthenia gravis, preferably in a human patient.

Preferably, from 10 to 1000 micrograms of SEQ ID NO:3 (preferably from 50 to 750 micrograms, advantageously from 100 to 500 micrograms) and/or from 10 to 1000 micrograms of SEQ ID NO:5 (preferably from 50 to 750 micrograms, advantageously from 100 to 500 micrograms), values expressed in “epitope equivalent”, even if the peptides of SEQ ID NOs: 3 and 5 are coupled to a carrier protein, or between 50 and 3000 micrograms of SEQ ID NO:1 coupled to SEQ ID NO:2 or to SEQ ID NO:3 and/or between 50 and 3000 micrograms of SEQ ID NO:1 coupled to SEQ ID NO:4 or to SEQ ID NO:5 are injected in a human patient (with MG).

Preferably, this pharmaceutical composition (used in immunization/vaccination against MG) further comprises an adjuvant.

In this pharmaceutical composition, the tryptophan residue in position 8 of SEQ ID NO:2 or SEQ ID NO:3 is not modified chemically; alternatively, this residue is alkylated on the free carbon of its indole group, preferably with a 2,4,6-trimethoxybenzyl group.

One aspect of the present invention is a method of production of a medicinal product for use in the treatment or prevention of myasthenia gravis comprising the steps of:
obtaining the carrier protein, which is SEQ ID NO:1;
activating the carrier protein (SEQ ID NO:1) by means of a heterobifunctional crosslinking agent so as to cause a plurality of —NH2 groups to react with the bifunctional crosslinking agent;
separating the activated carrier protein from the crosslinking agent that has not been incorporated;
contacting the activated carrier protein with one of the peptide epitopes selected from the group consisting of SEQ ID NO:2, SEQ ID NO:3, SEQ ID NO:4 and SEQ ID NO:5 so as to cause this peptide epitope to react with the carrier protein activated by the crosslinking agent;
separating the carrier protein coupled to several peptide epitopes from the unreacted substrates and the reaction by-products.

Preferably, in this method, the crosslinking agent is reactive for an —NH2 group and an —SH group and the peptide epitope is SEQ ID NO:3 or SEQ ID NO:5.

Preferably, this method further comprises a step of lyophilization of the carrier protein conjugated to the peptide epitope and/or a step of dissolving the carrier protein coupled to several of the epitopes in an aqueous solution comprising a specified pH buffer.

Advantageously, this buffer ensures a pH of 3.0, 3.5, 4.0, 4.5, 5.0, 5.5, 6.0, 6.5, 7.0, 7.5, 8.0 or 8.5. The above values are preferably ±0.2, even more preferably ±0.1, or even closer to the targeted value. Thus, a pH of 3.0 preferably signifies a pH above 2.9 and below 3.1. The preferred pH buffers are selected from the group consisting of acetate pH 4.5, N-(2-acetamido)iminodiacetic acid pH 6.5, Bis-Tris pH 6.0, CHES (2-(N-cyclohexylamino)ethane acid) pH 9.5, citric acid pH 3.2 (or 4.0 or 5.5), imidazole pH 8, glycine pH 3.0 (i.e. glycine-HCl), HEPES (4-(2-hydroxyethyl)-1-piperazine ethane sulphonic acid) pH 7.5, MES (2-(N-morpholino)ethanesulphonic acid) pH 6.2, MOPS (3-(N-morpholino)propanesulphonic acid) pH 7.0, PIPPS (piperazine-N,N′-Bis (3-propanesulphonic) acid) pH 3.7 and phosphate, pH 5.0.

### Detailed Description of an Embodiment of the Invention

To increase the efficacy of the treatment by immunization (vaccination) by means of peptides that are mimetic of the antigen receptor (antigen receptor mimetic, ARM) of myasthenia gravis (MG) in a patient, the inventors had the intuition of using a carrier protein, SEQ ID NO:1, not normally used in combination with peptide epitopes, especially peptide epitopes that are also hydrophobic.

Grafting of the peptides of SEQ ID NOs:2 and 4 on SEQ ID NO:1 was very complicated, in particular owing to the excessive change in the solubility properties of SEQ ID NO:1 when it was conjugated (coupled) with these peptides. The options for overcoming these changes are limited in the case of pharmaceutical compositions, since certain solvents cannot be used, or their concentration must be limited; for example, acetonitrile cannot be used at concentrations of 50% or higher when there is a lyophilization step, as this presents risks of explosion.

However, despite the initial setbacks, the inventors finally succeeded in obtaining a method of coupling (conjugation) that overcame all these difficulties. The inventors succeeded in grafting (coupling, conjugating) several peptide epitopes on a molecule of SEQ ID NO:1, in particular SEQ ID NO:3 and SEQ ID NO:5.

Thus, a first aspect of the present invention is a pharmaceutical composition comprising SEQ ID NO:3, SEQ ID NO:5 and/or a mixture of the two.

A related aspect is a pharmaceutical composition comprising one or more peptide epitopes selected from the group consisting of SEQ ID NO:2, SEQ ID NO:3, SEQ ID NO:4 and SEQ ID NO:5, these peptide epitopes being conjugated (coupled) covalently on one or more free NH2 residues of SEQ ID NO:1.

Advantageously, a plurality of one of the peptide epitopes is conjugated (coupled) on several free NH2 residues of SEQ ID NO:1, for example on at least 4, 5, 6, 7 or 8 free NH2 residues of SEQ ID NO:1, but preferably on fewer than 20, 19, 18, 17, 16, 15 or 14 free NH2 residues of SEQ ID NO:1.

Preferably, coupling was carried out by means of a heterobifunctional crosslinking agent, especially when several peptide epitopes were coupled.

Preferably, coupling was carried out by means of a crosslinking agent that is reactive for an amine group and a sulphydryl group, advantageously sulpho-GMBS. In that case the peptide epitopes to be coupled were SEQ ID NO:3 or SEQ ID NO:5; in practice the epitope of SEQ ID NO:3 was coupled to a molecule of SEQ ID NO:1, and the epitope of SEQ ID NO:5 was coupled to another molecule of SEQ ID NO:1, although, in a variant, the two epitopes could have been coupled to the same molecule of SEQ ID NO:1.

This pharmaceutical composition is advantageous for vaccination of a patient, preferably selected from the group consisting of human (Homo sapiens), dog (Canis vulgaris), horse (Equus caballus) and a member of the camel family (Camelus sp.), for example for use in the treatment of MG.

Advantageously, between 30 and 3000 micrograms, preferably between 150 and 2000 micrograms, advantageously between 300 and 1500 micrograms of SEQ ID NO:1 coupled to SEQ ID NO:2 or to SEQ ID NO:3, and/or to SEQ ID NO:4 or to SEQ ID NO:5 is administered to the (human) patient in an injection.

In practice, these values are adapted depending on the patient and/or are normalized as a function of their relative content of peptide epitopes, a higher coupling valence signifying injection of a smaller amount of the conjugate.

Advantageously, the patient receives several injections of SEQ ID NO:1 (conjugated with SEQ ID NO:2 or 3 and/or with SEQ ID NO:4 or 5) in the course of time, for example a second injection after some weeks, followed by a third after some weeks, or even more injections in the course of time.

Good results were obtained using from 10 to 1000, preferably from 50 to 750, for example from 100 to 500 micrograms of SEQ ID NO:2 or SEQ ID NO:3, and using from 10 to 1000, preferably from 50 to 750, for example from 100 to 500 micrograms of SEQ ID NO:4 or SEQ ID NO:5; these peptide epitopes being coupled (conjugated) covalently to SEQ ID NO:1.

Advantageously, the pharmaceutical composition is used or combined with an adjuvant such as emulsions, oligonucleotides having CpG units and aluminium salts. The preferred adjuvants are emulsions (oil-in-water and water-in-oil), or oligonucleotides having CpG units.

Preferably, the pharmaceutical composition is, or will be with a view to administration thereof to the patient, in aqueous solution comprising a buffer ensuring a specified pH.

This pH buffer is advantageously of 3.0, 3.5, 4.0, 4.5, 5.0, 5.5, 6.0, 6.5, 7.0, 7.5, 8.0 or 8.5. The above values are preferably ±0.2, even more preferably ±0.1, or even closer to the targeted value. Thus, a pH of 3.0 preferably signifies a pH above 2.9 and below 3.1.

The preferred pH buffers are selected from the group consisting of acetate pH 4.5, N-(2-acetamido)iminodiacetic acid pH 6.5, Bis-Tris pH 6.0, CHES (2-(N-cyclohexylamino)ethane acid) pH 9.5, citric acid pH 3.2 (or 4.0 or 5.5), imidazole pH 8, glycine pH 3.0, HEPES (4-(2-hydroxyethyl)-1-piperazine ethane sulphonic acid) pH 7.5, MES (2-(N-morpholino)ethanesulphonic acid) pH 6.2, MOPS (3-(N-morpholino)propanesulphonic acid) pH 7.0, PIPPS (piperazine-N,N′-Bis (3-propanesulphonic) acid) pH 3.7 and phosphate, pH 5.0.

Preferably, the acids/bases of these pH buffers are used at a concentration from 5 to 50 mM, preferably from 20 to 50 mM. The pH values are preferably within a range of ±0.2; thus, “pH 5.0” signifies from 4.8 to 5.2, even if a pH of strictly 5.0 is preferred (in this example explaining the range of pH). Advantageously, an additive is combined with the pH buffer, preferably selected from arginine and glutamine (50 mM each), trimethylamine N-oxide (500 mM), Tween®20 (1%; w:v), trehalose (500 mM) and glycerol (20%; v:v).

In this pharmaceutical composition, the tryptophan residue in position 8 of SEQ ID NO:2 or SEQ ID NO:3 is not necessarily modified chemically.

However, advantageously, the tryptophan residue in position 8 of SEQ ID NO:2 or SEQ ID NO:3 is alkylated on the free carbon of its indole group, for example with a 2,4,6-trimethoxybenzyl group, as in patent EP2004217.

A related aspect of the present invention is a method of production of a medicinal product for use in the treatment or prevention of MG, comprising the steps of:
obtaining the carrier protein, which is SEQ ID NO:1;
activating this carrier protein by means of a heterobifunctional crosslinking agent so as to cause a plurality of —NH2 groups to react with this bifunctional crosslinking agent;
separating the activated carrier protein from the crosslinking agent that has not been incorporated;
contacting this activated carrier protein with one of the peptide epitopes selected from the group consisting of SEQ ID NO:2, SEQ ID NO:3, SEQ ID NO:4 and SEQ ID NO:5 so as to cause these peptide epitopes to react with this carrier protein activated by the crosslinking agent;
separating the carrier protein coupled to several peptide epitopes from the unreacted substrates and the reaction by-products;
optionally, putting the carrier protein coupled to several peptide epitopes in aqueous solution comprising a buffer ensuring a specified pH for this aqueous solution.

This pH buffer is advantageously of 3.0, 3.5, 4.0, 4.5, 5.0, 5.5, 6.0, 6.5, 7.0, 7.5, 8.0 or 8.5. The above values are preferably ±0.2, even more preferably ±0.1, or even closer to the targeted value. Thus, a pH of 3.0 preferably signifies a pH above 2.9 and below 3.1. The preferred pH buffers are selected from the group consisting of acetate pH 4.5, N-(2-acetamido)iminodiacetic acid pH 6.5, Bis-Tris pH 6.0, CHES (2-(N-cyclohexylamino)ethane acid) pH 9.5, citric acid pH 3.2 (or 4.0 or 5.5), imidazole pH 8, glycine pH 3.0 (i.e. glycine-HCl), HEPES (4-(2-hydroxyethyl)-1-piperazine ethane sulphonic acid) pH 7.5, MES (2-(N-morpholino)ethanesulphonic acid) pH 6.2, MOPS (3-(N-morpholino)propanesulphonic acid) pH 7.0, PIPPS (piperazine-N,N′-Bis (3-propanesulphonic) acid) pH 3.7 and phosphate, pH 5.0.

As stated above for the aspect of the invention relating to the pharmaceutical composition, the pH values are preferably within a range of ±0.2; thus, “pH 5.0” signifies from 4.8 to 5.2, even if a pH of strictly 5.0 is preferred (in this example explaining the range of pH).

Advantageously, an additive is combined with the pH buffer, preferably selected from arginine and glutamine (50 mM each), trimethylamine N-oxide (500 mM), Tween®20 (1%; w:v), trehalose (500 mM) and glycerol (20%; v:v)

Preferably, in this method, the crosslinking agent is reactive for an —NH2 group and an —SH group and the peptide epitope is SEQ ID NO:3 or SEQ ID NO:5.

Advantageously, especially when organic solvents have been used, this method further comprises a last step of lyophilization of the carrier protein conjugated to the peptide epitope. The lyophilization step is not preferred when the method is carried out without organic solvents.

Thus, the present invention also relates to the peptide epitopes selected from the group consisting of SEQ ID N0:2, SEQ ID N0:3, SEQ ID NO:4 and SEQ ID NO:5 (preferably SEQ ID NO:3 or SEQ ID NO:5) coupled to SEQ ID NO:1 and obtainable by this method.

Advantageously, both SEQ ID NO:3 coupled to SEQ ID NO:1, and SEQ ID NO:5 coupled to SEQ ID NO:1, and obtained by this method, are administered to a patient with MG.

Other features and advantages of the present invention can be seen from the non-limiting description given hereunder, and by referring to the examples.

### EXAMPLES

Of course, the present invention is not in any way limited to the embodiments described above, and many modifications can be made to it while remaining within the scope of the accompanying claims.

### Example 1: Synthesis of SEQ ID NO:1 and of the Peptides of SEQ ID NO:2-4

SEQ ID NO:1 was obtained by fermentation of a particular strain of Escherichia coli; the sequence has been modified so that the protein (mature; SEQ ID NO:1) is secreted in the periplasmic space. The protein was, for example, obtained according to what is described in patent application BE2021/5137, filed on 26 Feb. 2021. The protein is recovered and purified by filtration and by chromatography, for example as described in application BE2021/5138, filed on 26 Feb. 2021. None of the reagents used is of animal origin. The protein obtained is of a purity compatible with clinical use.

The peptides of SEQ ID NO:2, 3, 4 and 5 were obtained by solid phase synthesis. However, other methods of synthesis, or of fermentation, are possible. In practice, the C-teminal end of the peptide is fixed to a polymer support and the peptide chain is formed and then extended to the N end by repetition of coupling cycles; the carboxy end of the amino acid to be incorporated was activated, and then coupled to the amino end of the peptide fixed to the resin. The amino groups are protected by the Fmoc group and the other potentially reactive functional groups are protected by other groups. When the amino acid is grafted to the peptide to be extended, after removing the amino acids in excess and after the washing steps, the Fmoc group is cleaved by adding piperidine. Cleavage of the resin and deprotection of the functional groups of the side chains of the amino acids are carried out by adding trifluoroacetic acid (TFA).

In certain cases, the tryptophan residue of SEQ ID NO:2 or SEQ ID NO:4 is alkylated on the indole group by adding an amide-Tmob to the TFA.

After cleavage of the resin, deprotection, purification (e.g. reversed-phase HPLC) and filtration (0.45 μm), the peptides were analysed by mass spectrometry (MALDI-TOF) and by HPLC. The steps of HPLC are carried out each time by means of a mixture of water (0.1% TFA) with an acetonitrile gradient (0.1% TFA), and UV detection (215 and 280 nm). The purity must be greater than 90%, or even 95%. In practice, the inventors obtained peptides with a purity of 97%, or even greater.

### Example 2: Coupling by Means of EDC, with Respect to Glutaraldehyde

First, the inventors tried to perform coupling using the method described in patent EP2004217, but applied on the peptides of sequences SEQ ID NO:2 and SEQ ID NO:4 to be coupled on SEQ ID NO:1 in place of KLH.

This type of coupling seems to work, but is not satisfactory in practice. In fact, the inventors observed that the molecule obtained was difficult to purify or sterilize and there was a lack of uniformity from batch to batch. One possible explanation is that the hydrophilic character of the unmodified protein is transformed excessively when a peptide is coupled to it, making this carrier protein unusable for the downstream treatments. Thus, the protein of SEQ ID NO:1 is useful as a carrier, but this usefulness is not evident in the context of the coupling of peptide epitopes.

However, the inventors nevertheless tried to overcome this difficulty, by choosing another coupling agent, 1-ethyl-3-[3-dimethylaminopropyl] carbodiimide hydrochloride (EDC); available for example from Pierce. This is an agent for coupling carboxyl groups to primary amines. EDC reacts with the carboxyl group to form a reaction intermediate, O-acylisourea. If this intermediate does not react with an amine, it undergoes hydrolysis, regenerating the carboxyl group.

For this purpose, the inventors developed a method of coupling in one step, and a method of coupling in two steps.

In the method in one step, such as the method based on glutaraldehyde, the carrier protein (SEQ ID NO:1), EDC and the epitope of SEQ ID NO:2 or of SEQ ID NO:4 are collected together in the same container with magnetic stirring, and then the precipitate is recovered and purified on Sephadex™ G50 prior to lyophilization.

In the method with two steps, SEQ ID NO:1 is first activated by being brought into contact with EDC, and then the epitope of SEQ ID NO:2 or of SEQ ID NO:4 is added prior to separation, purification and lyophilization.

With respect to glutaraldehyde, the reaction product is usable, even if there are still unusable aggregates, synonymous with losses of yield, and a significant portion of SEQ ID NO:1 in solution, synonymous with protein that has not reacted (or has, but insufficiently), i.e. a second loss of yield. Moreover, the efficacy of coupling varies considerably from batch to batch, and the solubility of the conjugate is mediocre, or even insufficient, which in addition complicates sterilization by filtration.

The yield was mediocre in the case of the method with one step: 12% for grafting of SEQ ID NO:2 and 30% for grafting of SEQ ID NO:4. However, the yield increases, for example to 60%, when coupling is carried out in two steps (SEQ ID NO:1 with SEQ ID NO:4). The molar distribution ratio SEQ ID NO:1 vs SEQ ID NO:2 or SEQ ID NO:4 is less than 2 for the method of coupling in one step, and is greater than 2 for the method of coupling in 2 steps.

### Example 3. Coupling of the CRM197 Protein with a Peptide, Using GMBS

The inventors dissolved 25 mg of CRM197 carrier protein (SEQ ID NO:1) in 2.5 ml of an aqueous solution. The carrier protein is from a batch compatible with clinical use and has a content of endotoxin below the limit of detection.

The inventors weighed 19 mg of sulpho GMBS (Pierce; reference 22324; N-y-maleimidobutyryl-oxysulphosuccinimide ester), then added 2.5 ml of a conjugation medium (100 mM of phosphate-buffered saline PBS and 10 mM of ethylenediaminetetraacetic acid EDTA) and 2.5 ml of the CRM solution. This mixture is stirred with magnetic stirring for 1 h at 4° C.

The inventors purified the activated SEQ ID NO: 1 by passing through a column of Sephadex™ G50 equilibrated with PBS buffer, pH 7.2, monitoring the recovery of SEQ ID NO:1 at 280 nm.

The inventors transferred the fraction(s) containing the absorption peak at 280 nm (CRM protein-GMBS) to a bottle containing SEQ ID NO:3 (25 mg, dissolved in DMSO) and stirred this by magnetic stirring for 2 h at 4° C. The conjugate formed between SEQ ID NO:1 and SEQ ID NO:3 precipitates. After centrifugation, the pellet is dissolved in acetonitrile.

The inventors added 1 ml of 1M cysteine, stirring for 30 minutes.

The inventors transferred the reaction mixture to a 50 ml Falcon® tube; after centrifugation, the pellet is separated and taken up in a water:acetonitrile mixture (70:30; w:w).

The inventors carried out purification on a Sephadex™ G50 column, again monitoring the absorption at 280 nm.

The inventors lyophilized the purified conjugate.

All the above steps were carried out in conditions compatible with subsequent clinical use. Studies of stability were then conducted, with a view to clinical use. The powder, stored at a temperature of −20° C. and of +5° C., remains stable for several months. It is resistant to accelerated ageing (stress at 25° C.) for at least 14 days (measurements by SDS-PAGE, MALDI-TOF, capillary electrophoresis, unchanged, and infrared (FTIR) to evaluate any degradation of chemical residues or of secondary structure).

The analysis reveals that SEQ ID NO:1 reacted very predominantly with the peptide of SEQ ID NO:3; a yield of 64% was obtained. Besides a good yield, the molar ratio between SEQ ID NO:1 and SEQ ID NO:3 is above 8. Values of 13 were even recorded for this coupling yield. Even higher ratios were obtained, but at the expense of a lower coupling yield (e.g. 36 and 47%).

In practice, the method of coupling with heterobifunctional agents, especially when it is carried out in two steps, even allows a coupling rate of more than 50 μg of peptide to 100 μg of SEQ ID NO:1.

SEQ ID NO:1 that has been coupled to several peptides of SEQ ID NO:3 or 5 (see below) is easily differentiated (e.g. by electrophoresis) since its molecular weight changes from 58 kDa to ˜78 KDa if 10 peptides are coupled, or even ˜90 kDa if more peptides are coupled to a molecule of SEQ ID NO:1; e.g. 13 or 14.

### Example 4

The same protocol is applied for the peptide of SEQ ID NO:5, also with a very good coupling yield and a very high molar ratio.

### Example 5

The peptide from example 3 (182 μg, or an equivalent of 60 μg of SEQ ID NO:3) is combined with the peptide from example 4 (182 μg, or an equivalent of 60 μg of SEQ ID NO:5). An adjuvant is added to this mixture or to a placebo. The active principle or the placebo is injected in a cohort of human patients with MG, in double blind conditions. In practice, 3 sequential injections are carried out (week 1, 5 and 13). Then the same protocol is designed for a second cohort of patients, but with even more active principle. Finally, the study is continued in “open” conditions, so as to evaluate the long-term tolerability and efficacy of the treatment. At each injection, the patient is closely monitored for any side-effects, firstly in the hospital with a high level of supervision and then at home. Blood samples are taken for immunogenicity assays.

The data generated make it possible to conclude that there is good tolerability (the active principle does not cause more side-effects than the placebo) and efficacy of the treatment.

1. A pharmaceutical composition comprising a peptide epitope selected from SEQ ID NO:3, SEQ ID NO:5, and a mixture of the two.

2. A pharmaceutical composition comprising one or more peptide epitopes selected from the group consisting of SEQ ID NO:2, SEQ ID NO:3, SEQ ID NO:4, and SEQ ID NO:5, said peptide epitopes being coupled covalently to one or more free —NH2 residues of SEQ ID NO:1.

3. The pharmaceutical composition of claim 2, in which a plurality of the one or more peptide epitopes selected from the group consisting of SEQ ID NO:2, SEQ ID NO:3, SEQ ID NO:4, or SEQ ID NO:5 is coupled to a plurality of free —NH2 residues of SEQ ID NO:1.

4. The pharmaceutical composition of claim 3, in which the plurality of the one or more peptide epitopes selected from the group consisting of SEQ ID NO:2, SEQ ID NO:3, SEQ ID NO:4, or SEQ ID NO:5 is coupled to at least 4 free —NH2 residues of SEQ ID NO:1.

5. The pharmaceutical composition of claim 3, in which the plurality of the one or more peptide epitopes selected from the group consisting of SEQ ID NO:2, SEQ ID NO:3, SEQ ID NO:4, and SEQ ID NO:5 is coupled to fewer than 20 free —NH2 residues of SEQ ID NO:1.

6. The pharmaceutical composition according to claim 2, in which coupling is via a heterobifunctional crosslinking agent.

7. The pharmaceutical composition according to claim 6, in which coupling is via a crosslinking agent that is reactive for an amine group and a sulphydryl group.

8. A method for immunization of a patient, the method comprising administering the pharmaceutical composition of claim 1 to the patient.

9. A method for treating myasthenia gravis the method comprising administering the pharmaceutical composition of claim 1 to a patient in need thereof.

10. The pharmaceutical composition according to claim 1, comprising from 10 to 1000 micrograms of SEQ ID NO:3 and/or from 10 to 1000 micrograms of SEQ ID NO:5.

11. The pharmaceutical composition according to claim 8, further comprising a vaccination adjuvant.

12. The pharmaceutical composition according to claim 2, in which the tryptophan residue at position 8 of SEQ ID NO:2 or SEQ ID NO:3 is not modified chemically.

13. The pharmaceutical composition according to claim 2, in which the tryptophan residue at position 8 of SEQ ID NO:2 or SEQ ID NO:3 is alkylated on the free carbon of its indole group.

14. A method of production of a medicinal product for use in treating or reducing the likelihood of developing myasthenia gravis, comprising the steps of:
obtaining a carrier protein, which is SEQ ID NO:1;
activating said carrier protein with a heterobifunctional crosslinking agent so as to cause a plurality of —NH2 groups to react with said heterobifunctional crosslinking agent;
separating activated carrier protein from unincorporated crosslinking agent;
contacting said activated carrier protein with one or more peptide epitopes selected from the group consisting of SEQ ID NO:2, SEQ ID NO:3, SEQ ID SEQ ID NO:5, and combinations thereof, so as to cause a plurality of said one or more peptide epitopes to react with said activated carrier protein;
separating the activated carrier protein coupled to the plurality of the one or more peptide epitopes from unreacted substrates and reaction by-products.

15. The method according to claim 14, in which:
the crosslinking agent is reactive for an —NH2 group and an —SH group, and
the one or more peptide epitopes comprises SEQ ID NO:3 or SEQ ID NO:5.

16. The method according to claim 14, further comprising a step of lyophilization of the activated carrier protein conjugated to the plurality of the one or more peptide epitopes.

17. The method according to claim 14, further comprising a step of dissolving the activated carrier protein coupled to the plurality of the one or more peptide epitopes in an aqueous solution comprising a buffer so as to ensure a specified pH for said aqueous solution.

18. The pharmaceutical composition of claim 3, in which the plurality of the one or more peptide epitopes comprises a plurality of the same one of the peptide epitopes.

19. The pharmaceutical composition of claim 8, in which the patient is selected from the group consisting of a human (Homo sapiens), a dog (Canis vulgaris), a horse (Equus caballus), and a member of the camel family (Camelus sp.).

20. The method according to claim 14, in which the one or more peptide epitopes comprises a plurality of the same one of the peptide epitopes.


SEQUENCE LISTING (5 sequences)

SEQ ID NO: 1 - protein, 535 aa, Artificial Sequence
  Modif. Diphteria toxin
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 16 aa, artificial sequence
  RhCA67-16
  MISC_FEATURE (8)..(8): Potentially hydrocarbonated on indole, such as 2,4,6-trimethoxybenzyl
FNSTIIGWIPAKPHIN

SEQ ID NO: 3 - protein, 17 aa, artificial sequence
  RhCA67-16-Cys
  MISC_FEATURE (8)..(8): Potentially hydrocarbonated on indole, such as 2,4,6-trimethoxybenzyl
FNSTIIGWIPAKPHINC

SEQ ID NO: 4 - protein, 17 aa, artificial sequence
  RhCA611
YFSRIIQKQFGHVNNGK

SEQ ID NO: 5 - protein, 18 aa, artificial sequence
  RhCA611-001-Cys
YFSRIIQKQFGHVNNGKC